(19) United States
(12) Patent Application Publication
(10) Pub. No.: US 2016/0194679 A1
(43) Pub. Date: Jul. 7, 2016

(54) Process for Producing Protein Concentrate or Isolate and Cellulosic Thermochemical Feedstock From Distillers Grains

(71) Applicants: Ian Mackay, Eden Praire, MN (US); Karl Greden, Hinckley, MN (US)

(72) Inventors: Ian Mackay, Eden Praire, MN (US); Karl Greden, Hinckley, MN (US)

(21) Appl. No.: 14/971,998

(22) Filed: Dec. 16, 2015

Related U.S. Application Data
(63) Continuation-in-part of application No. 14/591,904, filed on Jan. 7, 2015.

(51) Int. Cl. C12P 21/06; C12P 19/04

(52) U.S. Cl. CPC C12P 21/06; A23K 1/06; A23K 1/1826; C12P 19/04

(57) ABSTRACT

A process for treating distillers grains to produce a high value protein product and a cellulosic residue both from distillers grains. The high value protein product is useful as a protein supplement or feed for livestock and poultry and the cellulosic residue has value as a feedstock for a thermochemical process unit for the production of a biofuel.

### CROSS-REFERENCE TO RELATED APPLICATIONS

This application is a Continuation-In-Part of application Ser. No. 14/591,904 filed Jan. 7, 2015 which is based on Provisional Application 61/924,678 filed on Jan. 7, 2014.

### BACKGROUND OF THE INVENTION

This invention relates to a process for treating distillers grains to produce a high value protein product and a cellulosic residue both from distillers grains. The high value protein product is useful as a protein supplement or feed for livestock and poultry and the cellulosic residue has value as a feedstock for a thermochemical process unit for the production of a biofuel.

### BACKGROUND OF THE INVENTION

A substantial amount of research and development is being done to reduce our dependency on petroleum-based energy and to move us toward more sustainable and environmentally friendly energy sources, such as wind energy, solar energy, and biomass. The conversion of biomass into transportation and other fuels is of great interest for reducing reliance on fossil fuels. Many biomass conversion technologies employ thermochemical processes, such as pyrolysis and gasification that have relatively high capital and operating costs. In particular, sourcing and preparing biomass feedstocks, such as wood and agricultural residues, such as corn stover and soybean hulls, for pyrolysis or gasification, typically result in marginal production economics.

Another process for reducing the reliance on fossil fuels that has met with some degree of commercial success is “Ethanol Fermentation” which results in an alcohol-based alternative fuel by fermenting and distilling starch crops that can be converted into simple sugars. Typical starch-containing feedstocks include sorghum, corn, barley and wheat, with corn being most preferred. There are two main types of corn ethanol production: dry milling and wet milling In dry milling process the entire corn kernel is ground into flour, which is often referred to as “meal”. The meal is then slurried by adding water to form a mash. Enzymes are added to the mash to convert starch to dextrose, a simple sugar. Ammonia is typically added to control the pH and as a nutrient for the yeast, which is added later. The mixture is processed at high-temperatures to reduce bacteria levels then transferred and cooled in fermenters where yeast is added and conversion from sugar to ethanol and carbon dioxide begins. After the process is complete, everything is transferred to distillation columns where the ethanol is separated from “stillage”. The stillage is then processed to produce a nutritious livestock feed.

In a wet-milling process the corn grain is steeped in a dilute mixture of sulfuric acid and water in order to separate the grain into its various components. The resulting slurry mix then goes through a series of grinders to separate out the corn germ. Corn oil is a by-product and is extracted and sold. The remaining components of fiber, gluten and starch are separated by any suitable conventional separation technology non-limiting examples of which include screens, and hydroclonic and centrifugal separators. The gluten protein is dried and filtered to make a corn gluten-meal co-product which is sold as a poultry feed ingredient. The steeping liquor produced is concentrated and dried with the fiber being sold as corn gluten feed to the livestock industry. The corn starch and remaining water can then be processed by one of three ways: 1) fermented into ethanol, through a similar process as in dry milling, 2) dried and sold as modified corn starch, or 3) made into corn syrup.

The drying milling corn ethanol process is of particular interest for the present invention because of the resulting distillers grains by-product. There are various types of distiller's grains, wet and dry. Wet distillers grains (WDG) contain primarily unfermented grain residues (protein, fiber, fat and up to about 70 wt. % moisture). WDG have a shelf life of four to five days. Owing to the water content, WDG transport is usually economically viable within only about 125 miles of the ethanol production facility. Dried distillers grains with solubles (DDGS) is WDG that have been dried with the concentrated thin stillage to about 10 to 12 percent moisture. DDGS have an almost indefinite shelf life and may be shipped to practically any market regardless of its proximity to an ethanol plant. Drying is costly because it requires further energy input. DDGS is commonly packaged and traded as a commodity product.

While the above processes have met with varying degrees of commercial success there is still a need for improving the efficiency and economics of such processes.

### SUMMARY OF THE INVENTION

In accordance with the present invention there is provided a process for producing a protein product and a cellulosic product suitable as a feedstock for thermochemical processing from distillers grains containing a protein component, which process comprises:
a) combining: i) distillers grains having a protein content, and ii) an effective amount of a protease enzyme in a reaction vessel;
b) heating the combination of distillers grains and protease enzyme at a temperature of about 20° to about 80° C. for about 60 to about 150 minutes resulting in a liquid fraction containing hydrolyzed proteins, and a solids fraction comprised of protein-lean cellulosic distillers grains;
c) separating said liquid fraction from said solids fraction;
d) spray drying said liquid fraction resulting in spray dried hydrolyzed protein product;
e) drying said solids fraction and collecting the protein-lean cellulosic distillers grains.

In a preferred embodiment, the distillers grains are pre-processed by milling to a particle size of less than about 0.5 mm.

In another preferred embodiment, the distillers grains are wet and are pre-processed by treating them with ultrasonic energy for an effective amount of time to improve protein accessibility.

In another preferred embodiment of the present invention the distillers grains are a by-product from a corn to ethanol process.

In another preferred embodiment of the present invention the hydrolyzed proteins are comprised of peptides and amino acids.

In yet another preferred embodiment of the present invention the protease enzyme is selected from the group consisting of serine proteases, threonine proteases, cysteine proteases, aspartate proteases, glutamic acid proteases, and metalloproteases.

Also in accordance with the present invention there is provided a process for producing a protein product and a cellulosic product suitable as a feedstock for thermochemical processing from distillers grains containing a protein component, which process comprises :
a) milling distillers grains to a particle size of less than about 0.5 mm;
b) treating the milled distillers grains with a basic aqueous solution having a pH of at least 11 thereby resulting in a basic solution containing extracted proteins from the distillers grains and a protein-lean cellulosic distillers grains solid product ;
c) separating the basic solution containing the extracted proteins from the cellulosic distillers grains solids;
d) spray drying the basic solution containing extracted proteins resulting in a protein concentrate or isolate;
e) drying said cellulosic distillers grains solids.

In a preferred embodiment, the milled distillers grains are subjected to an effective amount of ultrasonic energy capable of improving the accessibility of proteins of the distillers grains.

In another preferred embodiment the base is a mineral base preferably sodium hydroxide.

### BRIEF DESCRIPTION OF THE FIGURES

FIG. 1 hereof is a schematic of a preferred embodiment of the present invention wherein the wet distillers grains are hydrolyzed by use of an enzyme, then centrifuged to result in: i) an hydrolyzed protein concentrate or isolate and ii) a cellulosic feedstock material that can be used to produce a synthetic gas in a thermochemical process.

FIG. 2 hereof is a schematic of another preferred embodiment of the present invention wherein the distillers grains are milled, then extracted with a suitable basic aqueous solution to dissolve or separate a substantial fraction of the proteins. The resulting basic aqueous protein-containing solution is then separated from the resulting protein-lean cellulosic distillers grains solids that can be used a food ingredient or feed component for livestock.

### DETAILED DESCRIPTION OF THE INVENTION

Distillers grains used in the practice of the present invention can be from any source. There are two main sources of these grains, brewers and grain to ethanol fuel production facilities. When sourced from a brewing operation, the grains are often called brewers grains or spent brewers grains. The following description refers more particularly to a grains to ethanol fuel production facility. Consequently, the term “distillers grains” as used herein include brewers grains as well.

Corn is composed of about two-thirds starch, which is converted to ethanol and carbon dioxide during a distilling and fermentation process. The remaining nutrients in corn, such as protein, fat, minerals and vitamins, are concentrated in three different ways and end up as distillers grains or condensed distillers solubles. The major products resulting from fermentation and distillation are ethanol and whole stillage. The ethanol is typically denatured and sold as a gasoline additive. Whole stillage typically contains about 13 to 17 wt. % solids and is comprised primarily of small particles of corn that did not get converted to ethanol. This whole stillage is typically centrifuged resulting in thin stillage and wet distillers grains (WDG). WDG is a more concentrated form of whole stillage and contains about 35 wt. % solids after leaving a centrifuge. Since it contains mostly solids, it typically must be augured or conveyed to a drum drier to produce dried distillers grains. The thin stillage is primarily water containing about 4 to 5 wt. % solids. The thin stillage is evaporated down to a concentrated syrup in an evaporator and the condensed water can be recycled to a slurry tank holding milled grain and an enzyme. This condensed water is typically called backset and helps to conserve total water usage. The remaining liquid is concentrated (syrup) by evaporation and mixed with the wet distillers grains before entering a dryer. The syrup is approximately 28 to 30 wt. % solids and contains mostly protein and oils from the grain, preferably corn. The addition of syrup increases the nutritional value of the DDG. The mixture of syrup and wet distillers grains is generally dried to generate dried distillers grain with solubles (DDGS). DDGS is typically dried to a 10% moisture level.

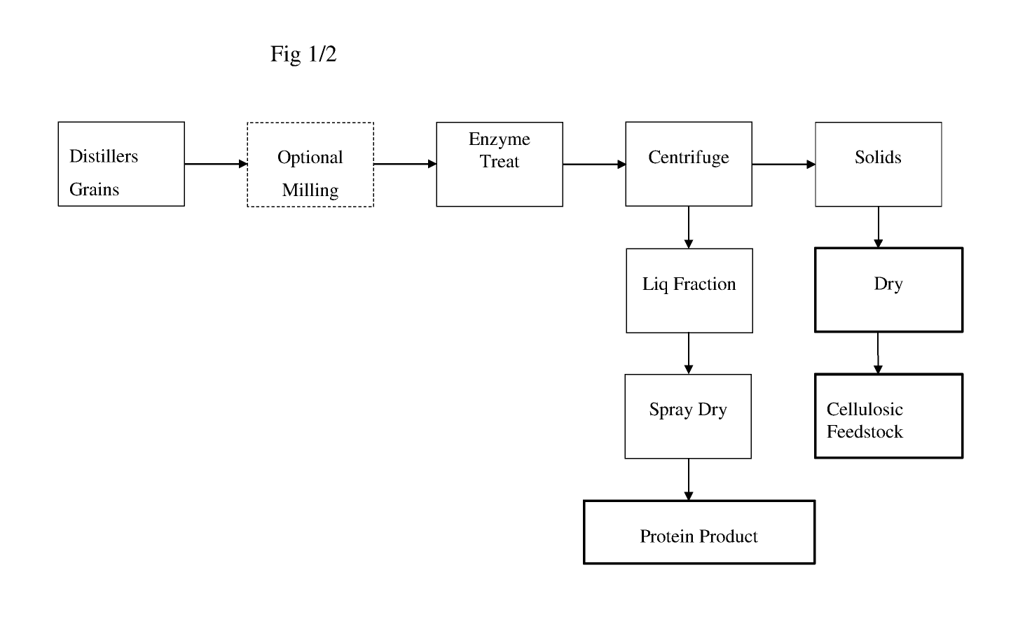

While the above grain to ethanol process has met with commercial success there is continuing development being done to squeeze additional profits from such processes by ways of finding more economical uses for the by-products, namely distillers grains. The present invention has accomplished this and can be better understood with reference to the figures hereof. FIG. 1 hereof is a preferred embodiment wherein distillers grains (wet or dry) are treated with an effective amount of a protease enzyme. FIG. 1 hereof shows an optional milling step in the event that they are not within an average particle size of about 0.25 mn to about 1 mm, preferably up to about 0.5 mm.

By effective amount of protease enzyme we mean at least that amount needed to reduce at least about 5% to about 12%, preferably from about 9% to about 11%, of the average protein chain length in the distillers grains to smaller chain peptides and amino acids. Any suitable protease enzyme can be used in the practice of the present invention. Non-limiting examples of protease enzymes that can be used in the practice of the present invention include serine proteases, threonine proteases, cysteine proteases, aspartate proteases, glutamic acid proteases, and metalloproteases. Aspartate and serine proteases are preferred, with serine being more preferred. The enzyme treated distillers grains are subjected to hydrolysis conditions to cause at least a fraction of the proteins of the distillers grains to hydrolyze, thus resulting in water soluble smaller chain materials, such as peptides and amino acids. The protease enzyme will preferably be used in an aqueous solution form of adequate concentration to provide the 0.5 to 2 wt. %, preferably from about 0.8 to 1.2 wt. %, based on the total weight of distillers grains being treated.

Hydrolysis conditions include: temperatures from about 10° C. to about 100° C., preferably from about 20° C. to about 80° C., more preferably from about 30° C. to about 70° C. and most preferably from about 40° C. to about 60° C.; and times from about 30 minutes to 180 minutes, preferably from about 60 minutes to about 150 minutes, and more preferably from about 90 minutes to about 130 minutes.

The resulting enzyme treated distillers grains mixture is conducted to a liquid/solids separation stage resulting in a liquid fraction comprised of water and hydrolyzed proteins and a predominantly solids fraction comprised of the remaining wet distillers grain having a substantially reduced level of proteins. It is preferred that the separation stage be comprised of a centrifuge. The liquid fraction is dried, preferably by spray drying to produce a hydrolyzed protein concentrate or isolate. The solids fraction, which is the remaining wet distillers grains is dried to produce a cellulosic residue product that is suitable as a fiber feed source or as feedstock for a thermochemical process that can convert it into a transportation or other fuel.

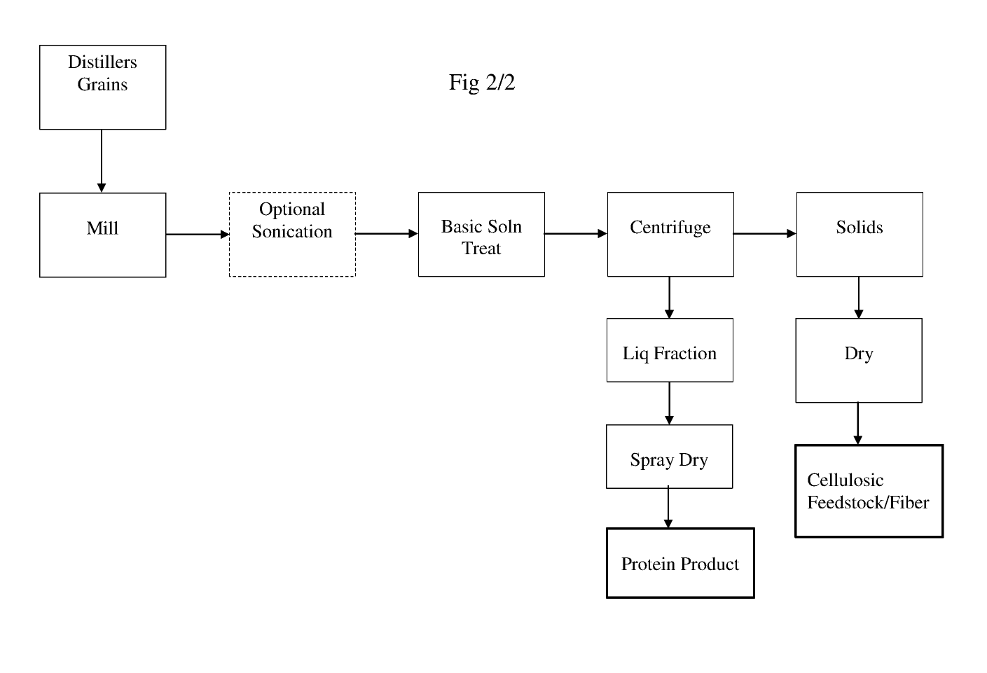

Reference is made to FIG. 2 hereof which represents another preferred embodiment for processing distillers grains to produce a protein concentrate or isolate product and a protein-lean distillers gain residue that can be used as a feed component for livestock or as feedstock for a thermochemical fuel. In this embodiment the distillers grains are milled to an average particle size from about 0.05 mm to about 0.5 mm, preferably from about 0.05 mm to about 0.3 mm. At least a fraction of the protein is extracted from the milled distillers grains with use of a basic aqueous solution at effective extraction conditions. The basic component of the will be a hydroxide of a metal selected from Groups 1 and 1 of the Periodic Table of the Elements. Preferred metals include sodium, potassium, magnesium and calcium, with sodium and potassium being the more preferred and sodium being the most preferred. By effective extraction conditions we mean extraction at a pH of 10 to 12, preferably at pH 10.5 to 11.5, more preferably a pH of 11; at a temperature range of about 20° C. to about 60° C.; and with a grains to basic solution ratio of 1:5 to 1:10.

The resulting basic distillers grains mixture is conducted to a separation zone wherein the fraction containing dissolved proteins is separated from a protein-lean cellulosic distillers grains residue fraction. It is preferred that the separation be done by centrifuge. The protein fraction is acidified with any suitable acid, preferably liquid form, to pH from about 4 to 6, preferably a pH of about 4.5 to 5.5 and spray dried resulting in a substantially dry protein product. The protein-lean cellulosic distillers grains residue is collected where it can be marketed as a livestock feed component or as a feedstock component for a subsequent thermochemical process, such as pyrolysis or gasification which can be used for the production of biofuel, preferably a transportation fuel, preferably a distillate fuel. The protein product obtained by the practice of the present invention will be a protein concentrate or preferably a protein isolate comprised of at 80 wt. % protein.

In the case of both enzyme and chemical processing of distillers grains, it preferred that the treated distillers brains be subjected to an effective amount of ultrasonic energy to improve the efficiency of the protein extraction portion of the process. The preferred effective ultrasonic energy input is from about 3 to about 30 Joules/gram of distillers grains with a frequency of about 40 kHz with about 3 to about 10 Joules/gram being preferred.

The following non-limiting examples are provided to show the various process various for practicing the instant invention.

### EXAMPLE 1

In a first preferred embodiment of the present invention the distillers grains are milled, either wet or dried to less than 0.5 mm wherein an effective amount of water is added so that the water to grain ratio is 10:1. The resulting mixture is heated to a temperature of about 50° C. and pH of mixture adjusted to a value of 10.5 with use of a suitable base, preferably sodium hydroxide. The resulting solution is kept at this pH and temperature for about one hour wherein the pH is lowered to about 9 8-9.5 with use of a suitable acid, preferably hydrochloric acid. An effective amount of an alkaline protease, preferably alcalase, at a dosage of 10 mls/kg of protein is then added. The desired pH is maintained until a degree of hydrolysis of about 5 is reached, as measured by base addition. This produces a 90% extraction yield and a protein that is very dark brown in color. It also solubilizes double the amount of non-protein molecules from the grains as compared to the following treatment.

It will be understood that for this first embodiment the water to grain ratio can be from about 8:1 to about 15:1, the temperature can range from 45 to 65° C., the pH can range from about 8 to 9.5, the dosage of protease can range from about 1 to 100, and the degree of hydrolysis can range from about 2 to 10.

### EXAMPLE 2

In a second preferred embodiment of the present invention the distillers grains, wet or dried are milled to an average particle size of less than 0.5 mm wherein water is added until the water to grain ratio is about 10:1. The resulting mixture is then heated to a temperature of about 60° C. and the pH adjusted to about 9 with use of a suitable base material, preferably sodium hydroxide. An effective amount of an alkaline protease, such as alcalase, is then added at a dosage of 10 mls/kg of protein. The pH of 9 is maintained until a degree of hydrolysis of 5 is reached as measured by base addition. This produces an 85% extraction yield and a protein that is brown in color.

It will be understood that for this second embodiment the water to grain ratio can be from about 8:1 to about 15:1, the temperature can range from 45 to 65° C., the pH can range from about 8 to 9.5, the dosage of protease can range from about 1 to 100, and the degree of hydrolysis can range from about 6 to 14.

### EXAMPLE 3

In a third preferred embodiment of the present invention the instant invention is performed by milling the wet or dried grains to less than 0.5 mm then adding water so that the water to grain ratio is 10:1. The resulting mixture is then heated to a temperature of about 50° C. and the pH adjusted to a value of about 12 using a suitable base material, preferably sodium hydroxide. The resulting solution is maintained at this pH and temperature for about 1 hr, then lower the pH is lowered to about 9 with use of a suitable acid, preferably hydrochloric acid. This produces a 90% extraction yield and a protein that is very dark brown in color. It also solubilizes double the amount of non-protein molecules from the grains as compared to the second treatment.

It will be understood that for this third embodiment the water to grain ratio can be from about 8:1 to about 15:1, the temperature can range from 45 to 65° C., the pH can range from about 11 to 12.5.

### EXAMPLE 4

In a fourth preferred embodiment and after doing any of the above first through third preferred embodiments, the insolubles are removed and the pH is lowered to about 5 with use of a suitable acid material, preferably hydrochloric acid. The temperature is then adjusted to about 45-65° C. and an effective amount of a starch hydrolyzing enzyme, such as glucoamylase is added at to account for about 0.3% of total solids present. The pH and temperature is maintained for about 1 hr to hydrolyze the starch to glucose. The pH is then adjusted to a value of about 7. This does not result in any further protein yield as compared to the first through third embodiments but produces a liquid fraction with low starch content and high glucose content. The glucose can be easily separated from the desired protein in downstream membrane processing.

It will be understood that for this fourth embodiment the pH can be lowered to 4 to 6, the temperature can be in the range of about 45 to 65° C. and the enzyme can account for about 0.1 to 1.0 wt. % of total solid present.

### EXAMPLE 5

In a fifth preferred embodiment the procedure of the above first through third embodiments are followed, but after the milling and water addition steps, an effective amount of glucoamylase is added as described in the above fourth preferred embodiment. After the 1 hr reaction period, the pH is adjusted to a value as described in the first through third preferred embodiment. This does not result in any further protein yield as compared to the first through third embodiments but produces a liquid fraction with low starch content and high glucose content and it reduces acid and base usage.

### EXAMPLE 6

In a sixth preferred embodiment the milling step in the first through third preferred embodiments is replaced with a hydrocavitation, or ultrasonic, treatment step wherein the distillers grains are subjected to the ultrasound waves for 120 seconds (range of 30 to 120 seconds) at a power density of 1 W/mL (range of 0.3 to 2.56 W/mL) This produces a protein yield of 65%.

### EXAMPLE 7

In a seventh preferred embodiment the procedure of the above first through third embodiments is followed, but after the milling and water addition steps an ultrasound step is conducted as described in the above sixth preferred embodiment. This produces a protein yield of 65%.

### EXAMPLE 8

In a more preferred embodiment of the present invention the distillers grains, distillers grains, either wet or dried are milled to less than 0 5mm and an effective amount of water is added so that the water to grain ratio is 10:1. The resulting mixture is heated to about 60° C. and the pH of the resulting mixture is adjusted to a value of about 9 with use of a suitable basic material, preferably sodium hydroxide. An effective amount of a suitable alkaline protease, preferably alcalase, at a dosage of 10 mls/kg of protein is added. The pH and temperature are maintained until a degree of hydrolysis of about 10 is reached as measured by base addition. After a degree of hydrolysis of 10 is reached, the pH is lowered to about 4.5 with use of a suitable acid, preferably hydrochloric acid and the temperature adjusted to about 55° C. An effective amount of a starch hydrolyzing enzyme, preferably glucoamylase, at 0.3% of total solids present is added and the temperature and pH maintained substantially constant for about 1 hr to hydrolyze the starch to glucose. The pH is adjusted to about 8.5 after 1 hr.

It will be understood that for this eight embodiment the water to grain ratio can be from about 8:1 to about 15:1, the temperature can range from 45 to 65° C., the pH can range from about 8 to 9.5, the dosage of protease can range from about 1 to 100, and the degree of hydrolysis can range from about 8-14, and the enzyme can account for about 0.1 to 1.0 wt. % of the total solids present.

What is claimed is:

1. A process for producing a protein product and a cellulosic product suitable as a feedstock for thermochemical processing from distillers grains containing a protein component, which process comprises:
a) milling said distillers grains to an average particle size of less than or equal to 0.5 mm.
b) mixing said milled distillers grains with an effective amount of water to provide a an aqueous mixture wherein the water to grain weight ratio is about 10:1;
c) adjusting the pH of the aqueous mixture with an aqueous basic solution to a pH of about 10.5;
d) heating said pH adjusted aqueous mixture to a temperature of about 50° C. and keeping it at that temperature for about one hour;
e) adjusting the pH of the heated aqueous mixture with an aqueous acid solution to a pH of about 9;
f) adding an effective amount of a protease enzyme at a dose of about 10 mls/kg to the heated pH adjusted aqueous mixture of step e) above;
g) maintaining the pH of the mixture of step f) above until the degree of hydrolysis of proteins of about 5 is reached, thereby resulting in a liquid fraction containing hydrolyzed proteins, and a solids fraction comprised of protein-lean cellulosic distillers grains;
h) separating said liquid fraction from said solids fraction;
i) spray drying said liquid fraction resulting in spray dried hydrolyzed protein product; and
j) drying said solids fraction and collecting the protein-lean cellulosic distillers grains.

2. The process of claim 1 wherein the distillers grains are wet and are pretreated with ultrasonic energy.

3. The process of claim 1 wherein the distillers grains are a by-product from a corn to ethanol process.

4. The process of claim 1 wherein the aqueous basic solution is sodium hydroxide and the aqueous acid solution is hydrochloric acid.

5. The process of claim 1 wherein the distillers grains are a by-product from a brewery process.

6. The process of claim 1 wherein the hydrolyzed proteins are comprised of peptides and amino acids.

7. The process of claim 1 wherein the protease enzyme is selected from the group consisting of serine proteases, threonine proteases, cysteine proteases, aspartate proteases, glutamic acid proteases, and metalloproteases.

8. The process of claim 7 wherein the protease enzyme is an alkaline protease.

9. A process for producing a protein product and a cellulosic product suitable as a feedstock for thermochemical processing from distillers grains containing a protein component, which process comprises:
a) milling distillers grains to a particle size of less than about 0.5 mm;
b) mixing said distillers grains with water to that the ratio of water to distillers grains is about 8:1 to 15:1;
c) treating the milled distillers grains with a basic aqueous solution effective to result in the aqueous mixture having a pH of about 8 to 9.5 thereby resulting in an aqueous basic mixture containing a liquid fraction comprised of extracted proteins from the distillers grains and a solids fraction comprised of protein-lean cellulosic distillers grains;
d) heating said aqueous mixture of c) above to a temperature of about 45° C. to 65° C. and keeping it at that temperature for about one hour;
e) adding an effective amount of an alkaline protease at a dosage of about 1 to 100 mls/kg of protein of said distillers grains;
f) maintaining the temperature and pH until the degree of hydrolysis of about 8 to 14 is reached;
g) removing at least a fraction of any insolubles;
h) adjusting the pH of the heated aqueous mixture with an aqueous acid solution to a pH of about 5.5 while maintaining a temperature of about 45 to 65° C.;
i) adding an effective amount of starch hydrolyzing enzyme at a level of about 0.1 to 1.0 wt. % of total solids. j) maintaining a temperature of about 45 to 65° C. and pH of about 5.5 for about one hour to result in hydrolyzing at least a fraction of the starch to glucose;
k) adjusting the pH to about 8.5;
l) separating said liquid fraction from said solids fraction;
m) spray drying said liquid fraction resulting in spray dried hydrolyzed protein product; and
o) drying said solids fraction and collecting the protein-lean cellulosic distillers grains.

10. The process of claim 9 wherein the basic solution is a aqueous hydroxide solution of a metal selected from the group consisting of sodium, potassium, calcium and magnesium.

11. The process of claim 10 wherein the basic solution is sodium hydroxide solution.

12. The process of claim 9 wherein the distillers grains are wet and are pretreated with ultrasonic energy.

13. The process of claim 9 wherein the protease enzyme is an alkaline protease enzyme.

14. The process of claim 13 wherein the alkaline protease enzyme is alcalase.

15. The process of claim 9 wherein the distillers grains are a by-product from a corn to ethanol process.

16. The process of claim 9 wherein the distillers grains are a by-product from a brewery process.

17. The process of claim 9 wherein the hydrolyzed proteins are comprised of peptides and amino acids.
